(19) United States
(12) Patent Application Publication
(10) Pub. No.: US 2025/0249249 A1
(43) Pub. Date: Aug. 7, 2025

(54) IMPLANTABLE STIMULATION SYSTEM WITH ECG DETECTION

(71) Applicant: THE ALFRED E. MANN FOUNDATION FOR SCIENTIFIC RESEARCH, VALENCIA, CA (US)

(72) Inventor: Anthony C. Ng, Santa Clarita, CA (US)

(21) Appl. No.: 18/942,294

(22) Filed: Nov. 8, 2024

Related U.S. Application Data
(60) Provisional application No. 63/548,673, filed on Feb. 1, 2024.

(51) Int. Cl. A61N 1/36; A61N 1/05; A61N 1/375

(52) U.S. Cl. CPC A61N 1/36053; A61N 1/0556; A61N 1/36139; A61N 1/3752

(57) ABSTRACT

A medical system, according to some examples, includes an implantable pulse generator (IPG); a lead electrically coupled to the IPG; and an electrode on the lead, the electrode being configured to provide a stimulation signal to, and receive a first electrocardiogram (ECG) signal from, tissue around the electrode, wherein the IPG comprises a current source configured to provide an alternating current through the lead to the electrode, and wherein the lead is configured to selectively receive the current from the current source or to transmit the first ECG signal.

### CROSS-REFERENCE TO RELATED APPLICATION

The present application claims priority to, and the benefit of, U.S. Provisional Application No. 63/548,673, filed on Feb. 1, 2024, the entire content of which is incorporated herein by reference.

### FIELD OF THE INVENTION

The present disclosure relates generally to a tissue stimulation apparatus and methods of using and making the same. In particular, the present disclosure relates to a stimulation system, which may include an implantable pulse generator, a stimulation lead, and/or a stimulation electrode (such as, e.g., a nerve cuff), wherein the stimulation system is configured to provide stimulation and to perform electrocardiogram (ECG) measurement.

### BACKGROUND

This background section is provided only for purposes of introducing certain background material relating to the present disclosure and, thus, is not an admission of prior art.

Various types of devices have been developed for implantation into the human body to provide various types of health-related therapies and/or monitoring. Examples of such devices, generally known as implantable medical devices (“IMDs”), include cardiac pacemakers, cardioverter/defibrillators, cardiomyostimulators, various physiological stimulators, including nerve, muscle, and deep brain stimulators, various types of physiological monitors, and drug delivery systems, just to name a few. For purposes of this application, reference will be made primarily to implantable neurostimulators, such as implantable pulse generators (“IPGs”). However, those of ordinary skill in the art will understand that the aspects, features, and principles described herein with reference to example embodiments may be applied to other implantable medical devices as well.

IPGs can be used in the context of neuromodulation therapy. In general, IPGs may include a hermetically sealed housing that houses stimulation circuitry, and a header which is mounted on the IPG housing. The header may include a connector, and the connector may be electrically coupled to electronics within the housing by way of feedthrough pins that extend from the header connector into the housing. The connector may define a receptacle adapted to receive a proximal end of one or more stimulation leads, and a stimulation electrode (e.g., a nerve cuff) disposed at a distal end of the stimulation lead and configured to provide an interface for transmitting electrical current from the IPG to an anatomical target, such as biological tissue (e.g., a peripheral nerve).

Stimulation electrodes may generally include a body including a biocompatible, pliable, electrically insulative body formed from a substrate material, such as, e.g., silicone, and at least one electrode contact (“contacts”) may be carried by the body. The electrode contacts may be formed from an electrically conductive material, such as, e.g., platinum-iridium, may be electrically coupled to the IPG stimulation circuitry via the lead, and may be partially embedded in (or otherwise attached to) the substrate so that at least a portion of the electrode contact can interface with the anatomical target.

Stimulation electrodes may have a variety of configurations to accommodate different applications (e.g., designed to provide stimulation for particular types of tissue, such as specific nerves). A non-exhaustive list of stimulation electrodes generally available in the prior art includes: cuff electrodes (e.g., nerve cuffs), as disclosed in U.S. Pat. No. 9,283,394; helical electrodes, as disclosed in U.S. Pat. No. 4,573,481; paddle electrodes (e.g., electrode arrays), as disclosed in U.S. Pat. Pub. No. 2012/0209285 A1; and linear electrodes (e.g., percutaneous electrodes), as disclosed in U.S. Pat. No. 8,650,747.

A nerve cuff electrode may be configured to stimulate the peripheral nervous system. Nerve cuffs can be configured to gently wrap around the epineural surface of a nerve trunk. A nerve cuff can be used to stimulate the hypoglossal nerve to treat obstructive sleep apnea or to stimulate the vagus nerve to treat epilepsy. Some nerve cuffs can include a cuff body that may be pre-shaped to a furled state, may be movable to a slightly unfurled state, and may be manually unfurled into a flat state during surgical implantation and placed on a nerve. Other nerve cuffs may include a cuff body that is substantially flat, and, in such cases, a physician may manually wrap the cuff body around a nerve and fasten together free ends of the cuff body using sutures, specialized clamps, or the like.

In an example application, a stimulation system incorporating at least some of the aforementioned elements may be used for stimulation of a peripheral nerve, such as the vagus nerve, for the treatment of epilepsy.

Individuals with epilepsy may experience changes in their cardiac function. In some cases, there may be an elevation in heart rate during the minutes to hours leading up to a seizure. Furthermore, individuals with depression generally have a higher resting heart rate, about 10 to 15 beats per minute higher than those without depression. It can be advantageous for an IPG that treats epilepsy or depression to also collect heart rate data to help predict the onset of seizures and to diagnose depression. The membrane potential at the heart's sinoatrial node (the SA node) varies between −60 mV at the resting membrane potential to +20 mV at peak depolarization and this voltage (potential difference) can be measured near a healthy human heart as the heart muscle contracts.

FIG. 1A depicts a graph showing an example ECG signal, and FIG. 1B depicts a graph showing an example of membrane potentials which occur over the course of two heart periods, and the underlying behavior of select ionic currents that physiologically govern the electrical behavior of the membrane potential voltages. Referring to FIGS. 1A and 1B, action potentials are considerably different between cardiac conductive cells and cardiac contractile cells. While both sodium ions (Na+) and potassium ions (K+) play a role, calcium ions (Ca2+) also play a role for both types of cells. Unlike skeletal muscles and neurons, cardiac conductive cells do not have a stable resting potential. Conductive cells contain a series of Na+ ion channels that allow a normal and slow influx of Na+ ions that causes the membrane potential to rise slowly from an initial value of about −60 mV up to about −40 mV. The resulting movement of Na+ ions creates a spontaneous depolarization (or prepotential depolarization) and brings the cell to threshold. At this point voltage-gated Ca2+ ion channels open and Ca2+ ions enters the cell, thereby forming the rising phase of the action potential and further depolarizing it at a more rapid rate until it reaches a value of approximately +5 mV. At this point, the Ca2+ ion channels close and voltage-gated K+ ion channels open, allowing outflux of K+ ions, resulting in repolarization. When the membrane potential reaches approximately −60 mV, the K+ ion channels close and voltage-gated slow Na+ ion channels open, and the prepotential phase begins again. This phenomenon explains the auto-rhythmicity properties of cardiac muscle.

Some implantable pulse generators (IPGs) may be able to extract heart rate from the small movements and acceleration caused by heart muscle contraction. However, this approach is susceptible to noises generated from movement of other unrelated body motions. Conversely, measuring electrical signals intrinsically generated by the heart near the heart during heart muscle contraction can eliminate at least some such noise issues.

The present disclosure relates to a stimulation system that is easy to use, simple to manufacture, and is capable of reliably detecting heart rate without significant noise and without the incorporation of additional components (e.g., without incorporating dedicated ECG electrode(s)). Processes for manufacturing implantable medical devices (IMDs) continually stand to be improved for the purposes of minimizing product size, lowering production costs, increasing product longevity, improving product performance, and streamlining associated surgical procedures, among other purposes. This is also true for IMDs, such as IPGs, stimulation leads, and nerve cuff electrodes.

### SUMMARY

The present disclosure relates to improvements in implantable stimulation systems. Some known stimulation systems either (a) do not include means for heart rate detection, (b) include means for detecting heart rate but are highly susceptible to interference/noise, or (c) include means for detecting heart rate but require complex equipment and fabrication and/or the inclusion of additional components that must be implanted.

Accordingly, the present disclosure is directed, in part, towards an improved stimulation system, which may include an implantable pulse generator (IPG), one or more stimulation leads, and one or more stimulation electrodes. According to an aspect, one or more existing electrodes (e.g., electrodes that are configured for functions other than for capturing ECG signals) in the IPG or in the stimulation electrodes (e.g., nerve cuffs), or other existing conductive elements of the stimulation system, may be configured as one or more ECG electrodes to detect and record electrical activities of the heart or other physiological parameters over a period of time, which may include, but are not limited to, a patient's ECG waveform with P, T, and U wave segments, QRS complex, heart rate, respiratory rate, etc. and, thus, to have an additional functionality. Therefore, the need of a dedicated ECG sensor (e.g., dedicated ECG electrode(s) configured only for capturing ECG signals) for heart rate detection in the stimulation system can be eliminated.

In another aspect, the present invention is directed towards a method for detecting heart rate data (e.g., electrocardiogram (ECG) data) using the aforementioned stimulation system.

When a stimulation system is utilized for stimulation of the vagus nerve, an IPG may be implanted on the left chest of a patient near the heart. The IPG may include an outer housing that contains stimulation circuitry, and the IPG housing may be manufactured from an electrically conductive and biocompatible material, such as e.g., titanium. In some examples, the IPG is hermetically sealed at least in part by the housing. As such, the IPG itself (e.g., one or more portions of the housing, such as at least part of the can electrode) may be configured to functionally perform as an ECG electrode.

Because the IPG and its constituent conductive components may be in proximity to the heart, the stimulation system may be able to sense electrical signals generated during heart muscle contraction more effectively compared to detection electrodes positioned farther from the heart.

The stimulation electrodes (e.g., the cuff electrodes at the vagus nerve), which are implanted further away from the heart and closer to the neck, can be configured to provide a reference potential (common mode or differential mode). In some examples, the IPG and the stimulation electrodes (e.g., cuff electrodes) can be configured to measure transthoracic impedance, which some studies show to have a direct correlation to the heart beat. As such, the IPG housing (also referred to as the “CAN”) and electrode(s) (e.g., CAN electrode on the IPG or cuff electrode) in connection therewith may each functionally perform ECG detection.

In some examples, an additional sensing and protection circuit connected to the electrode may be added to the IPG's circuit board to protect its electronics against external stressors, such as internal defibrillation (e.g., defibrillation generated by an implantable cardioverter defibrillator implanted in a patient), an external defibrillation, or an MRI. This circuit may also be configured to filter and amplify ECG signals against external signals, such as signals originating from defibrillation and MRI, before feeding the ECG signals into an analog-to-digital converter (ADC) of (electrically connected to) a microcontroller unit (MCU) of the IPG that is configured to record and analyze the ECG data. The ADC could be external to the MCU or integrated inside the MCU.

The magnitude of cardiac action potentials (electrical impulses) may be measured in mV or tenths of mV, and can be amplified and measured by the IPG's new ECG function if the IPG electrode(s), such as the IPG's metal housing, is implanted in the proximity of the heart.

In some examples, a medical system includes an implantable pulse generator (IPG); a lead electrically coupled to the IPG; and an electrode on the lead, the electrode being configured to provide a stimulation signal to, and receive a first electrocardiogram (ECG) signal from, tissue around the electrode, wherein the IPG comprises a current source configured to provide an alternating current through the lead to the electrode, and wherein the lead is configured to selectively receive the current from the current source or to transmit the first ECG signal.

In some examples, an implantable pulse generator (IPG) includes a driver configured to generate an alternating stimulation current; a conductive can configured to house electronic components of the IPG and to receive a first electrocardiogram (ECG) signal from tissue around the conductive can; a protection circuit configured to receive the first ECG signal and to remove or attenuate signals having voltages above a threshold voltage; a differential signal amplifier configured to receive the first ECG signal, receive a second ECG signal, and generate an output signal based on a difference between the first and second ECG signals; and a microcontroller configured to receive the output signal.

In some examples, a medical system includes an implantable pulse generator (IPG); a lead electrically coupled to the IPG; and a stimulation electrode on the lead, wherein the IPG comprises: a current source configured to provide an alternating current through the lead to the stimulation electrode; a conductive can configured to house electronic components of the IPG and to receive a first electrocardiogram (ECG) signal from tissue around the conductive can; and an ECG electrode electrically insulated from the conductive can and configured to receive a second ECG signal from tissue around the ECG electrode.

This Summary section introduces some features of nonlimiting and non-exhaustive examples of the present disclosure, and is not intended to limit the scope of the claims.

### BRIEF DESCRIPTION OF THE DRAWINGS

The drawings illustrate some features of nonlimiting and non-exhaustive example embodiments of the present disclosure.

FIG. 1A depicts a graph showing an example ECG signal.

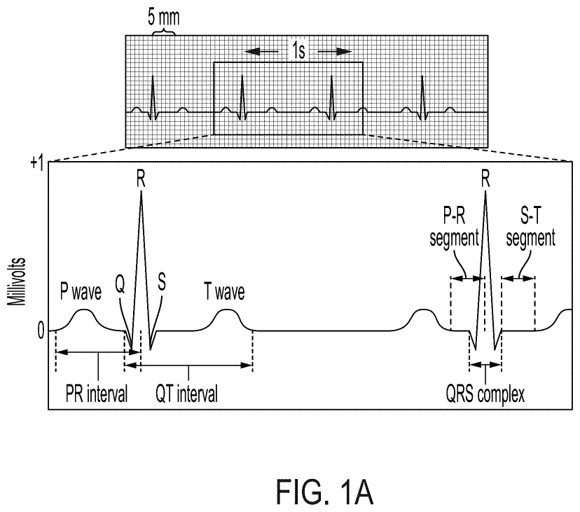

FIG. 1B depicts a graph showing the membrane potentials which occur over the course of two heart periods, and the underlying behavior of select ionic currents that physiologically govern the electrical behavior of the membrane potential voltages.

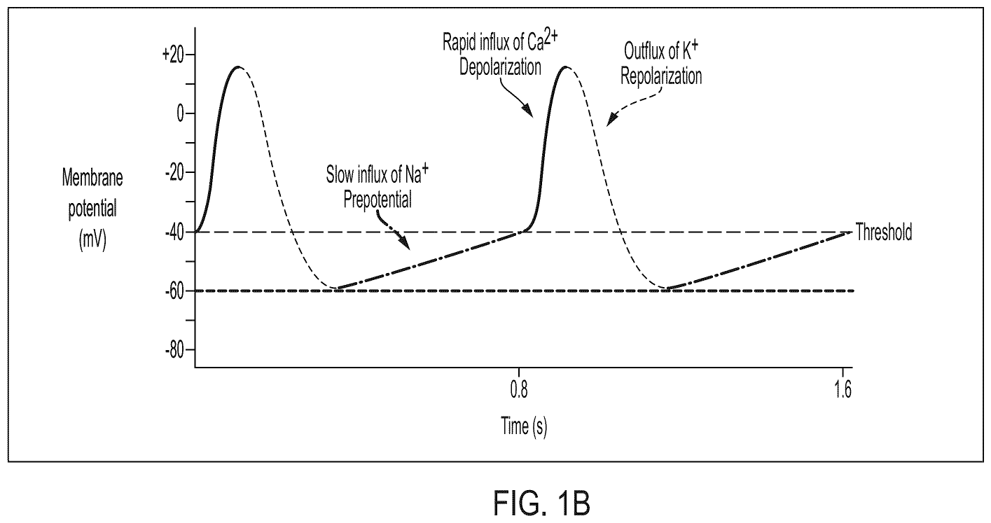

FIG. 2 depicts a stimulation and ECG sensing system according to some examples, where the stimulation and ECG sensing system is depicted as being implanted in a person, and some circuit electronics of the stimulation and ECG sensing system are schematically depicted.

FIG. 3A depicts the stimulation system of FIG. 2 according to some examples.

FIG. 3B depicts the electrode lead and the stimulation electrode of the stimulation system of FIG. 2 according to some examples.

FIG. 4 depicts a schematic view of some electronic components of the stimulation and ECG sensing system of FIG. 2, according to some examples, plus a bi-directional switch that connects the electrode to either a stimulation circuit or an ECG sensing circuit

FIG. 5 depicts a more detailed schematic view of some electronic components of the ECG sensing circuit from FIG. 4, according to some examples.

FIG. 6 depicts a waveform diagram of stimulation signals and electrocardiogram (ECG) signals on the lead of the stimulation system of FIG. 2, according to some examples.

FIG. 7 depicts another stimulation and ECG sensing system according to some examples, where the stimulation and ECG sensing system is depicted as being implanted in a person, and some circuit electronics of the stimulation and ECG sensing system are schematically depicted.

FIG. 8 depicts a schematic view of some electronic components of the stimulation and ECG sensing system of FIG. 7 according to some examples, with conductive contacts other than the lead as inputs for an ECG sensing circuit

FIG. 9 depicts the implantable pulse generator of the stimulation system of FIG. 7, according to some examples.

FIG. 10 depicts the implantable pulse generator of the stimulation system of FIG. 7, according to some other examples.

FIG. 11 depicts the implantable pulse generator of the stimulation system of FIG. 7, according to some other examples.

FIG. 12 depicts another stimulation system according to some examples, where the stimulation system is depicted as being implanted in a person, and some circuit electronics of the stimulation system are schematically depicted.

FIG. 13 depicts a schematic view of some electronic components of the stimulation system of FIG. 12, according to some examples.

### DETAILED DESCRIPTION

Nonlimiting and non-exhaustive example embodiments of systems and methods for providing stimulation and measuring electrocardiogram (ECG) data of a body (e.g., of a human patient) will now be described with reference to the drawings.

A stimulation system according to the present disclosure may include an implantable medical device (IMD), such as a pulse generator (IPG), one or more leads electrically coupled to the IPG, and one or more stimulation electrodes on the one or more leads that are configured to provide electrical stimulation to tissue (e.g., a nerve, such as the vagus nerve or the hypoglossal nerve). The stimulation system may be configured to detect electrocardiogram (ECG) data, to derive (e.g., determine or calculate) certain physiological parameters, e.g., heart rate, or to detect certain cardiac events, e.g., Atrial Fibrillation or, in some cases, respiratory rate, respiration phase, etc., using the ECG data. For example, ECG measurements may pick up, in addition to cardiac electrical signals, information about respiration rates and the respiration phase of the patient from contractions of the diaphragm muscle and intercostal muscles. The respiration rates and respiration phase measurements can be separated from the cardiac signals when the ECG measurement is processed and analyzed. In some examples, one or more electrically conductive components of the stimulation system, such as one or more of the stimulation electrodes and/or one or more conductive components of the lead or IPG (e.g., the can electrode of the IPG housing) may be configured to also function as ECG electrodes. By configuring such electrically conductive components of the stimulation system to both function as an ECG sensing electrode and to perform as an electrode as part of the stimulation circuit, the stimulation system can be configured to provide ECG sensing without increasing the number of components, complexity, and manufacturing cost of the stimulation system.

The stimulation system may be used, for example, in the context of neuromodulation therapy to treat various medical conditions, such as epilepsy or depression, by providing electrical stimulation to certain tissue, such as the vagus nerve. Such neuromodulation therapy may be based on the measured ECG data to improve the therapy. For example, the stimulation system may provide stimulation to the tissue in response to determining, based on measured

ECG data, that certain changes in the patient's heart rate indicate the onset of a seizure. In some examples, as explained above, depression can be diagnosed based, at least in part, on the patient's heart rate when the heart is at rest. Therefore, the stimulation system, which may be implanted in a patient for one or more reasons besides treating depression (e.g., for treating epilepsy) can also be used to diagnose and treat depression. Accordingly, by configuring the stimulation system to perform ECG sensing in addition to providing stimulation, the stimulation system can improve stimulation-based therapies and provide additional medical utility (e.g., assist in diagnosing or monitoring one or more medical conditions).

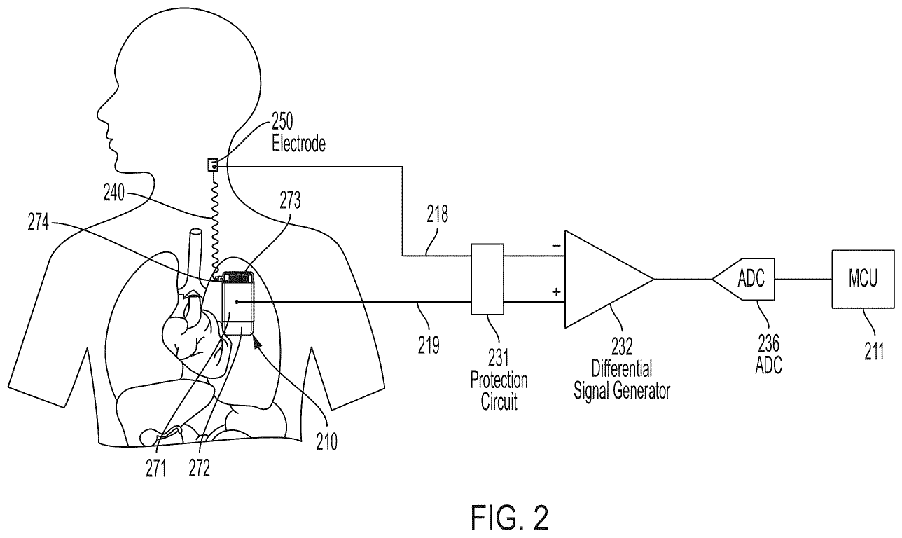

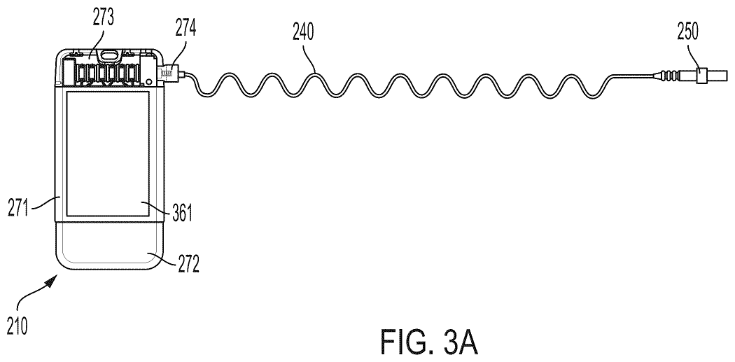

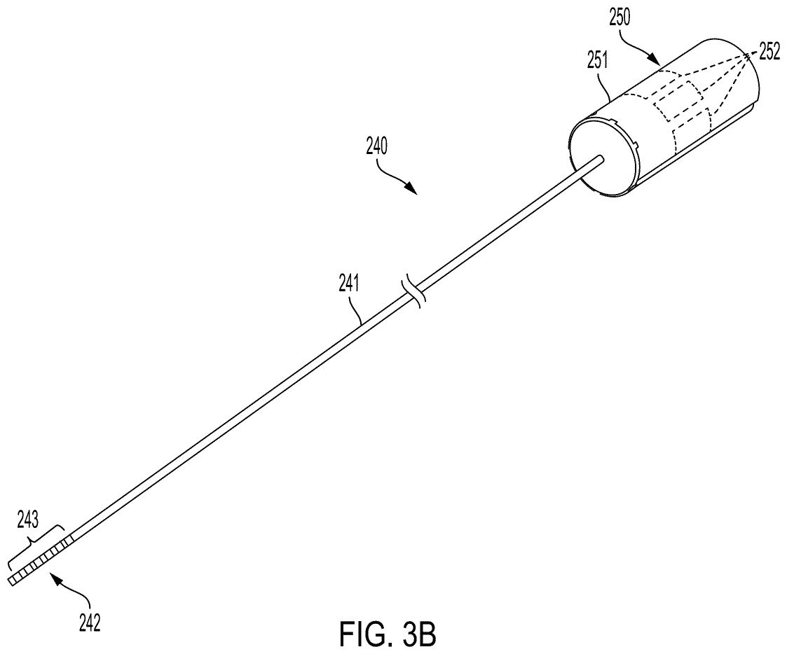

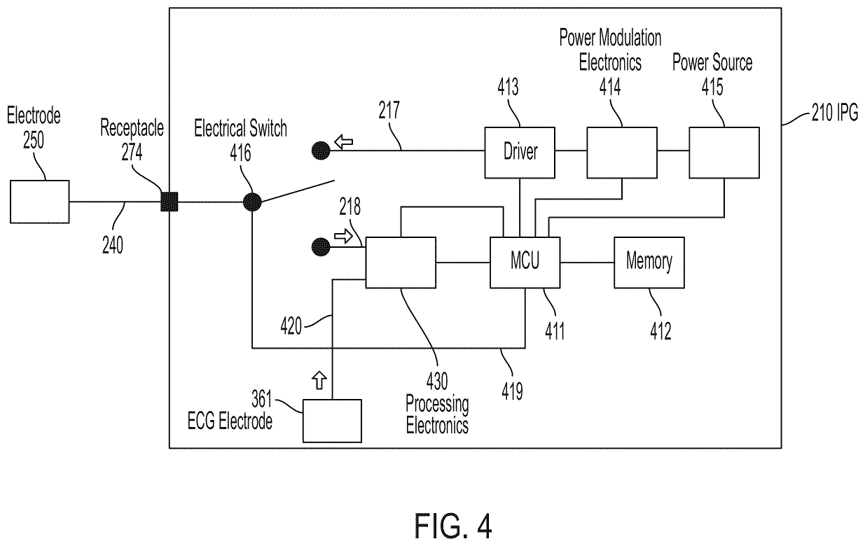

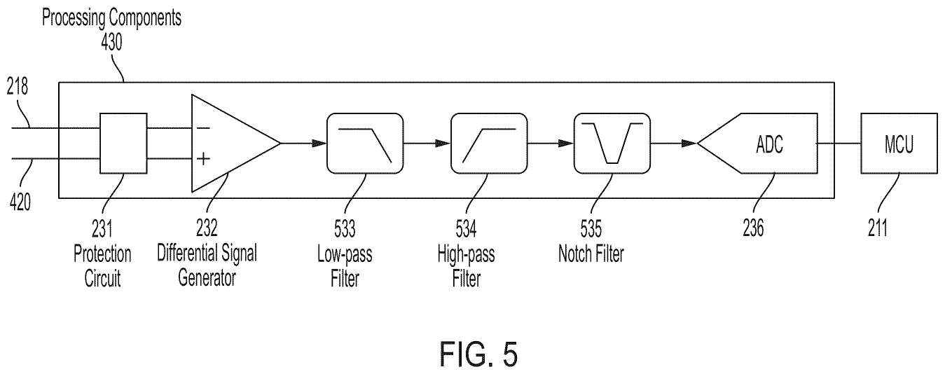

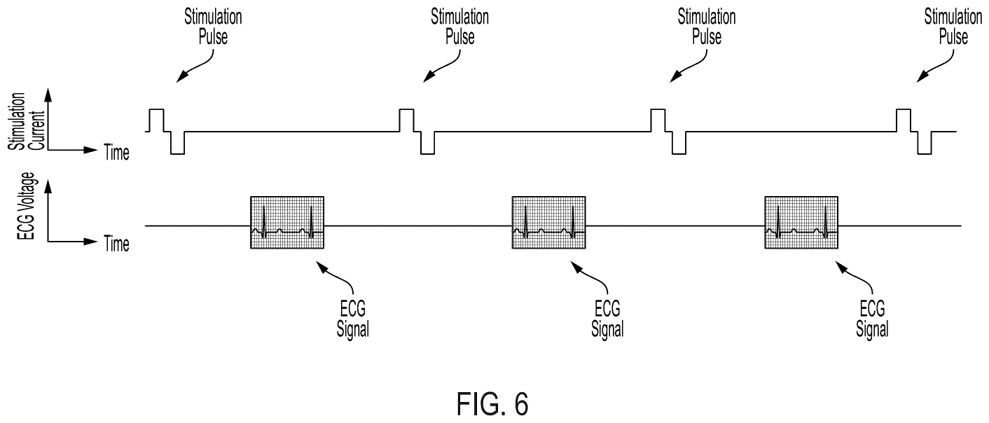

FIG. 2 depicts a stimulation and ECG sensing system according to some examples, where the stimulation system is designed to be implanted in a person, and some circuit electronics of the stimulation system are schematically depicted. FIG. 3A depicts the stimulation and ECG sensing system of FIG. 2 according to some examples. FIG. 3B depicts the electrode lead and the stimulation electrode of the stimulation system of FIG. 2 according to some examples. FIG. 4 depicts a schematic view of some electronic components of the stimulation and ECG sensing system of FIG. 2, according to some examples. FIG. 5 depicts a more detailed schematic view of some electronic components from FIG. 4, according to some examples. FIG. 6 depicts a waveform diagram of stimulation output signals and electrocardiogram (ECG) input signals on the lead of the stimulation system of FIG. 2, according to some examples.

Referring to FIGS. 2-6, the stimulation system may be configured to provide stimulation to tissue (e.g., to nerves, muscles, organs, etc.) and may include an implantable medical device (IMD), which is depicted in this example as an implantable pulse generator (IPG) 210. The stimulation system may also include a lead 240 electrically coupled to the IPG 210 and a stimulation electrode 250 on the lead 240. As explained in more detail below, the stimulation system may also be configured to perform ECG sensing.

The electrode 250 may be configured to stimulate tissue around the electrode 250, such as a particular type of tissue or a particular nerve. For example, the electrode 250 may be a cuff electrode configured to stimulate the vagus nerve or the hypoglossal nerve. In some examples, the electrode 250 includes a plurality of individually and differentially addressable electrode contacts, and the electrode 250 may include 2, 3, 4, 6, or more electrode contacts. In some examples where the electrode 250 includes a cuff electrode, the cuff electrode may include a plurality of the electrode contacts on a flexible band or strap that is configured to be wrapped around a nerve bundle (e.g., the vagus nerve). The electrode 250 may provide an interface for transmitting electrical current from the IPG to the nerve tissue. In some examples, the electrode 250 is at a distal end of the lead 240, as depicted in FIGS. 2 and 3. In some other examples, the electrode 250 is between the distal end and a proximal end of the lead 240. The stimulation system may include only one stimulation electrode (e.g., only the electrode 250), or it may include a plurality of stimulation electrodes. In some examples, the electrode is a nerve cuff having a plurality of distinct and electrically isolated conductive contacts.

In some examples, the stimulation system may include only one lead (e.g., only the lead 240), or the stimulation system may include a plurality of leads that are separately electrically coupled to the IPG 210. In the depicted example, the lead 240 is a single stimulation lead. In some other examples, the lead 240 may be a bifurcated lead with two or more sub-leads branching off of a common lead.

In the example of FIG. 3B, the stimulation electrode 250 is depicted as a nerve cuff electrode including a nerve cuff body 251, configured to at least partially encircle (e.g., wrap around) a nerve, and an array of electrode contacts 252 attached to an inside of the cuff body 251. The cuff electrode 250 may be configured such that, when the cuff body 251 encircles the nerve, the electrode contacts 252 contact the nerve. The lead 240 may include a lead body 241. A proximal end 242 of the lead body 241, which may be configured to couple to a header 273 of the IPG 210, may include a plurality of connector contacts 243 that may be respectively and independently electrically coupled to the electrode contacts 252 through the lead body 241.

The IPG 210 may include various electronic components configured to control operations of the stimulation system. In some examples, the IPG 210 includes a housing configured to contain the electronic components. The housing may be configured to be hermetically sealed and may include a metal can 271, the header 273 joined to the can 271, and an insulator portion 272 joined to the can 271. The header and 273 and the insulator portion 272 may be positioned at opposite sides (e.g., top and bottom sides) of the can 271. The insulator portion 272 may be, for example, a ceramic. In some other examples, the insulator portion 272 may be absent and may be replaced by an extension of the can 271 that covers (e.g., closes) one end of the can 271.

The can 271 may include a biocompatible conductive material, such as titanium, stainless steel, or another suitable metal. When the can 271 includes a conductive material, it can protect electronic components of the IPG 210 from some external magnetic fields. An ECG electrode 361 may be defined by at least part of the can 271 and may be configured to measure a first signal of the ECG signal input pair (the first ECG signal), as described in more detail below.

The header 273 may include a connector that is electrically coupled to electronics within the housing by way of feedthrough pins that extend from the header connector into the housing. The connector may define a receptacle (e.g., a port) 274 configured to receive the proximal end of the lead 240 such that electrical contacts of the lead 240 can be electrically connected to respective feedthrough pins of the header 273. In some examples, the header 273 includes an insulator material, such as a polymer, a glass, etc. The insulator portion 272 may include, for example, a ceramic material. By including insulator materials in the insulator portion 272 and the header 273, and positioning the insulator portion 272 and the header 273 on two sides (e.g., opposite sides) of the conductive can 271, some magnetic fields can propagate through insulator 272 to interact with certain components of the IPG 210 inside or near the insulator 272. Such magnetic fields may include, for example, an inductive charging field for recharging a rechargeable battery of the IPG 210 or a communication field for providing wireless communication between the IPG 210 and another device (e.g., a physician's controller device). In some examples, the insulator portion 272, is the same material as the can 271, such as a metal. For example, the insulator portion 272 may be omitted, and the can 271 may be enclosed at the end of the can 271 that the insulator portion 272 is depicted as being on.

Referring to FIG. 4, the electronic components of the IPG 210 may include a microcontroller unit (MCU) 411, a memory 412, a driver 413, power modulation electronics 414, a power source 415, an electrical switch 416, and processing electronics 430.

The MCU 411 may be operatively coupled to other electronic components of the IPG 210 and may be configured to control at least some operations of the IPG 210 in response to executing instructions stored in the memory 412. For example, the memory 412 may store computer-readable instructions that, in response to being executed by the MCU 411, cause the MCU 411 to perform operations described herein. The memory 412 may store ECG data for on-board analysis or to be transferred to another device (e.g., a clinician programmer device) for cloud-based analysis.

The lead 240 may be configured to selectively receive, along a first electrical path 217, an alternating current (a stimulation current) from the driver 413 to the electrode 250 or to transmit, along a second electrical path 218 different from the first electrical path 217, a second signal of the ECG signal input pair (the second ECG signal) to the processing electronics 430 and/or to the MCU 411. For example, the MCU 411 may be configured to controllably configure the lead 240 to have a first configuration, whereby the lead 240 is electrically coupled to the driver 413 along the first electrical path 217, or to have a second configuration, whereby the lead 240 is electrically coupled to the processing electronics 430 and/or to the MCU 411 along the second electrical path 218. In some examples, the lead 240 is configurable in only one of the first configuration or the second configuration at a time, and the MCU 411 is configured to change the lead 240 between the first configuration and the second configuration.

In the nonlimiting and non-exhaustive example depicted in FIG. 4, the IPG 210 includes the electrical switch 416, which is configured to selectively electrically connect the lead 240 to the first electrical path 217 or to the second electrical path 218. The MCU 411 may be operatively coupled to the electrical switch 416 and configured to controllably cause the electrical switch 416 to move between the first electrical path 217 and the second electrical path 218. For example, the MCU 411 may be configured to transmit one or more signals to the electrical switch 416 along a third electrical path 419 to control the electrical switch 416.

By configuring the lead 240 to be selectively couplable to the driver 413 or to ECG processing electronics 430, the electrode 250 can be used to both provide stimulation (when the lead 240 is coupled to the driver 413) and, alternatively, to transmit the second ECG signal to the ECG processing electronics 430, then to the MCU 411 (when the lead 240 is coupled to the processing electronics 430 then to the MCU 411). For example, the IPG 210 may be configured to perform time division multiplexing of stimulation pulses and ECG signals on the lead 240, as described in more detail below with reference to FIG. 6.

The driver 413 may be electrically couplable to the lead 240 along a first electrical path 217 and configured to provide an alternating stimulation current to the electrode 250 through the lead 240. The power source 415 may be configured to provide electrical power to the power modulation electronics 414, and the power modulation electronics 414 may be configured to modulate the received electrical power and to transmit the modulated power to the driver 413. The MCU 411 may be operatively coupled to the power source 415, the power modulation electronics 414, and the driver 413, and may be configured to controllably cause the power modulation electronics 414 and the driver 413 to convert electrical power from the power source 415 into an alternating current that has a set (e.g., predetermined) waveform, amplitude, and frequency.

In some examples, the power source 415 is a rechargeable battery that can, for example, be inductively recharged through the skin of a patient that the stimulation system is implanted in. In some other examples, the power source 415 may be a single-use, primary cell (non-rechargeable) battery or a power receiver device. In some other examples, the power receiver device may be configured to wirelessly receive power, convert the power into an electric current, and transmit the electric current to the power modulation electronics 414 without storing the electric current (e.g., in a battery).

The stimulation system may be configured to measure an ECG based on the first ECG signal captured by the ECG electrode 361 and the second ECG signal captured by the stimulation electrode 250. For example, the electrode 250 may be configured to function as a second ECG electrode. The ECG measurement may be based on at least two ECG signals (e.g., the first and second ECG signals with or without additional ECG signals obtained from additional ECG electrodes). The electrode 250 may be positioned further from the heart than the ECG electrode 361, for example, when the IPG 210 is implanted near the heart and the electrode 250 is positioned at the vagus nerve. Therefore, the first ECG signal captured by the ECG electrode 361 may have a greater amplitude than the second ECG signal captured by the electrode 250, and the second ECG signal may be used as a reference voltage (e.g., common mode or differential mode) that the first ECG signal can be compared to in order to at least partially isolate the ECG signal generated by the body's heart from other noise signals in the body. A common mode signal may include unwanted noise from outside the body that is picked up in both the first and second ECG signals. A differential mode signal may refer to the heart signal that may be obtained when the second ECG signal is subtracted out from the first ECG signal to generally obtain a signal generated by the heart.

The processing electronics 430 may be configured to receive the first and second ECG signals, to process the first and second ECG signals, and to output a processed ECG signal (e.g., a single ECG signal) based on the first and second ECG signals. The processing electronics 430 may be configured to output the processed ECG signal to the MCU 411 and/or to the memory 412 for storage and/or analysis. For example, the MCU 411 may be configured to determine (e.g., calculate) a heart rate, a respiratory rate, and/or a respiration phase based on ECG data received from the processing electronics 430.

The processing electronics 430 may be configured to receive the first ECG signal from the ECG electrode 361 along a fourth electrical path 420. The first electrical path 217, the second electrical path 218, the third electrical path 419, and the fourth electrical path 420 may be four separate electrical paths and, in some embodiments, may be electrically insulated (e.g., electrically disconnected) from each other.

Referring to FIG. 5, the processing electronics 430 may include a protection circuit 231, a differential to single-ended signal amplifier 232, a low-pass filter 533, a high-pass filter 534, a notch filter 535, and an analog-to-digital (ADC) converter 236. Although FIG. 5 depicts components of the processing electronics 430 as being electrically connected in a certain order, separated from each other, and separated from the MCU 211, this is only an example. The components of the processing electronics 430 may be electrically connected in any suitable order. At least some of the components of the processing electronics 430 may be combined into a single component that is configured to perform all of the functions of the said components. Any component of the processing components 430 may be divided and implemented as multiple separate components that are each configured to perform one or more of the functions or the original component, and that are collectively configured to perform all of the functions of said original component. In some examples, at least some of the components of the processing electronics 430 are integrated into the MCU 211, and the MCU 211 is configured to perform all of the functions of the said components.

The protection circuit 231 may be configured to receive the first and second ECG signals, to process the first and second ECG signals in a manner configured to protect other electronic components of the IPG 210, and to output the processed first and second ECG signals to the differential signal amplifier 232. The first and second ECG signals may include both signals generated by the heart and noise signals generated from other sources and that may be harmful to the electronics of the IPG 210. For example, the ECG electrode 361 and the electrode 250 may receive signals generated by a defibrillator, an ultrasound, an MRI induced field, eddy currents, etc., which could damage other electronics of the IPG 210. The protection circuit 231 may be configured to perform one or more operations to protect the electronics of the IPG 210 from such other signals.

In some examples, the protection circuit 231 is configured to block, or attenuate the voltage of, potentially harmful signals having a voltage above a threshold voltage value. For example, the protection circuit 231 may include circuit elements configured to limit the amplitude of the voltage of the processed first and second ECG signals. The threshold voltage value may be, for example, at least 500 V, 1000 V, 1500 V, 2000 V, 2500 V, 3000 V, 4000 V, or 5000 V. This can filter out high-voltage signals that originate, for example, from a defibrillator, an ultrasound, an MRI, or other sources of high-voltage electric currents. For example, voltages of external biphasic defibrillators can reach up to 2500 V, and monophasic defibrillators can be as high as 5000 V.

In some examples, the protection circuit 231 is configured to filter out electric signals based on their waveform. For example, the protection circuit 231 may include a low-pass filter configured to remove signals having a frequency below a threshold frequency value to filter out some noise signals. For example, the threshold frequency value may be at or below the lower end of frequencies for a normal resting heart, such as a value within the range of less than 0.9 Hz, 0.7 Hz, 0.5 Hz, 0.3 Hz, or 0.1 Hz. In some examples, the low-pass filter of the protection circuit 231 may be configured to filter out, for example, electrostatic discharge and lighting signals.

In some examples, the protection circuit 231 is configured to electrically disconnect itself from the first and second electrical paths 217 and 218 in response to a threshold event in order to reduce the risk of receiving potentially harmful signals. For example, the protection circuit 231 may include a blocking circuit configured to predict or determine that an electrical shock, an MRI, or another high-voltage event is about to occur and, in response, to electrically disconnect from the first and second electrical paths 217 and 218. The blanking circuit can therefore reduce the risk of receiving certain high-voltage signals. For example, the blanking circuit may be configured to operate in a magnetic resonance imaging (MRI) mode in which it causes the protection circuit 231 to electrically disconnect from the first and second paths 217 and 218 when an MRI is anticipated. For example, the IPG 210 may include a wireless transceiver (e.g., a radio, such as a Bluetooth radio), and the blanking circuit may be switched (e.g., by the MCU 411) into the MRI mode in response to the transceiver receiving a communication signal informing the IPG 210 that an MRI is about to occur. In some other examples, the MCU 411 may be configured to determine (e.g., based on the measured ECG) that the patient has flatlined (e.g., has no heartbeat) and may cause the protection circuit 231 to disconnect from the first and second paths 217 and 218 in anticipation that the patient will receive a defibrillation signal.

The differential signal amplifier 232 may be configured to receive the processed first and second ECG signals from the protection circuit 231 and to generate and output a single-ended signal amplified based on the processed first and second ECG signals. For example, the differential signal may be based on a difference between the processed first ECG signal and the processed second ECG signals. In some examples, the differential signal amplifier 232 is configured to subtract the second processed ECG signal from the first processed ECG signal to generate the single-ended signal. As discussed above, the electrode 250, from which the second processed ECG signal originates from, may be farther from the heart than the ECG electrode 361 and, thus, the processed second ECG signal may be used as a reference signal that the processed first ECG signal can be compared to, for example, in order to remove noises external to the body that are picked up by the first and the second ECG.

The differential signal amplifier 232 may also be configured to amplify the differential signal, which may allow the MCU 411 to obtain a more accurate ECG measurement. In some other examples, an amplifier separate from the differential signal amplifier 232 may be configured to amplify the differential signal generated and output by the differential signal amplifier 232. For example, the amplifier may be configured to receive the signal from the differential signal amplifier 232 and amplify the received differential signal, and output the amplified single-ended signal, for example, to the low-pass filter 533. The amplifier, whether integrated into the differential signal amplifier 232 or provided as the separate amplifier, may have a differential gain (e.g., close-loop differential gain) of at least 15 dB, 30 dB, 45 dB, or 60 dB, a common mode rejection ratio (input referred) of more than 20 dB, 40 dB, 50 dB, 55 dB, 60 dB, or 80 dB, and a power supply rejection ratio (input referred) of more than 50 dB, 65 dB, 80 dB, 95 dB, or 110 dB. The differential gain may refer to the amplification of the difference between the input at the positive (+) terminal and the negative (−) terminal of the differential signal amplifier 232. The common mode rejection ratio may relate to the ability of the amplifier to reject common mode noise or background noise that may be picked up by both ECG inputs. The power supply rejection ratio may relate to the ability of the amplifier to reject noise from a power source (e.g., noise from an on-board switching regulator inside the IPG 210 that can couple across a circuit board into a power rail of the processing components 430 on the same circuit board) that may be otherwise coupled into the amplified ECG signal.

The low-pass filter 533 and the high-pass filter 534 may be respectively configured to remove signals that are below an upper threshold frequency and above a lower threshold frequency. For example, the upper threshold frequency may be a frequency within the range of less than 5 Hz, 3 Hz, 1 Hz, 0.5 Hz, or 0.1 Hz, and the lower threshold frequency may be a frequency within the range of greater than 250 Hz, 275 Hz, 300 Hz, 325 Hz, or 350 Hz. Filtering out such signals may improve isolation of electric signals generated by the heart. The low-pass filter 533 and the high-pass filter 534 may allow for high-frequency noise and low-frequency artifacts to be filtered out from raw ECG signals. High-frequency noise may originate, for example, from power line interference, electrical equipment, or other sources of electromagnetic interference. These noises can obscure the QRS complexes and other important features of the ECG signal, making it difficult to accurately detect heartbeats and diagnose cardiac conditions. Low-frequency artifacts, on the other hand, may include baseline wander, movement artifacts, or respiratory interference. These artifacts can cause fluctuations in the ECG signal that mimic changes in heart activity, leading to incorrect interpretations of the data.

The notch filter 535 may be configured to attenuate or remove signals having a set frequency, such as 60 Hz, or within a relatively narrow frequency band. The notch filter 535 may be used to filter out signals caused by a power source, such as the power source of a building that the patient is in.

The ADC (analog-to-digital converter) 236 may be configured to convert an analog ECG signal, received from other components of the processing electronics 430, into a digital ECG signal and to transmit the digital ECG signal to the MCU 211. In the depicted example, the ADC 236 is a component of the processing electronics 430 and is separate from the MCU 211. In some other examples, the ADC 236 forms part of the MCU 211.

Referring to FIG. 6, the MCU 211 may be configured to control the configuration of the lead 240 such that both stimulation pulses to the electrode 250 and ECG signals from the electrode 250 can travel along the lead 240, for example, via a time-division multiplexing operation. Each stimulation pulse may, for example, be a bi-phasic pulse provided by the driver 413, and the stimulation pulses may be provided at a set first frequency. FIG. 6 depicts the stimulation pulses and the ECG signals, and the stimulation pulses and the ECG signals may travel along the same electrical path in the lead 240, although the stimulation pulses and the ECG signals may travel in the lead 240 in different directions and different times.

In some examples, the MCU 211 is configured to cause the lead 240 to be electrically connected to the second electrical path 218 only at times between when two adjacent stimulation pulses are provided so that the stimulation therapy is not interrupted by the ECG detection. The MCU 211 may cause the electrical switch 416 to switch back and forth between the first electrical path 217 and the second electrical path 218 at a set second frequency, which may be referred to as a sampling frequency. The sampling frequency may be substantially equal to (e.g., within 5%, 3%, or 1%) the first frequency, or the sampling frequency may be greater than or less than the first frequency. Normal ECG signals have a frequency band between 0.05 and 100 Hz. In some examples, the sampling frequency may be equal to or greater than 1 Hz, 5 Hz, 10 Hz, 25 Hz, 50 Hz, 100 Hz, 150 Hz, 200 Hz, 250 Hz, 300 Hz, 500 Hz, or 1000 Hz. Sampling frequencies between 10 Hz and 25 Hz can capture typical QRS complex from a healthy heart, and sampling frequencies of at least 250 Hz can exceed the Nyquist sampling frequency.

In some examples, the MCU 211 may cause the electrical switch 416 to switch from the first electrical path 217 to the second electrical path 218 for a period of time between two adjacent stimulation pulses. For example, between each adjacent pair of stimulation pulses from among a plurality (e.g., 2, 3, 4, 10, or more) of stimulation pulses, the MCU 211 may cause the electrical switch 416 to switch from the first electrical path 217 to the second electrical path 218 one or more times. In some other examples, between some adjacent pairs of stimulation pulses from among the plurality of stimulation pulses, the MCU 211 may cause the electrical switch 416 to switch from the first electrical path 217 to the second electrical path 218, and the MCU 211 may maintain the electrical switch 416 at the first electrical path 217 between some other adjacent pairs of stimulation pulses from among the plurality of stimulation pulses.

By configuring the stimulation electrode 250 and a portion of the can 271 to also function as ECG electrodes, the stimulation system can be provided with ECG electrodes without including dedicated ECG electrodes (e.g., electrodes that function solely for ECG detection). Therefore, manufacturing cost and complexity of stimulation systems according to some examples of the present disclosure can be reduced.

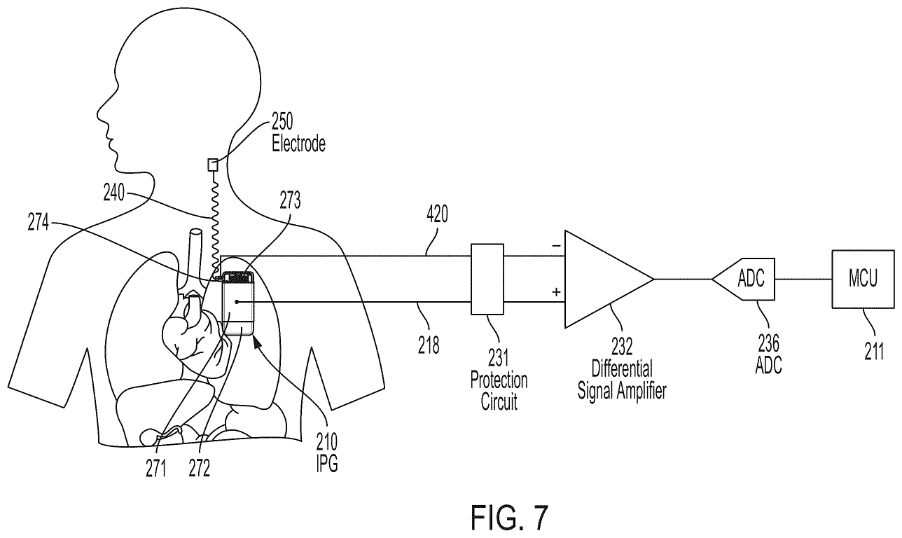

Although the ECG electrode 361 has been described as including at least part of the can 271, the present disclosure is not limited thereto. For example, another portion of the stimulation system such as an electrode or conductive element on the header 273, the insulator portion 272, or the lead 240 may be configured as an ECG electrode instead of, or in addition to, the ECG electrode 361. In some examples, one or more selected from among at least part of the can 271, a conductive element(s) on the header 273, and a conductive element(s) on the insulator portion 272 may be collectively configured to functionally perform as a single ECG electrode. Also, although the stimulation electrode 250 has been described as being configured as an ECG electrode, the present disclosure is not limited thereto, and multiple discrete and electrically separated electrodes on the stimulation system (e.g., on the IPG 210 and/or the lead 240) may instead be used, as described in more detail below with reference to FIGS. 7-12. In some examples, two or more of distinct electrically conductive contacts on the electrode 250 (e.g., on a cuff electrode) may be configured either as dedicated ECG electrodes or as dual ECG and stimulation electrodes that use time-division multiplexing techniques describes herein, and such ECG electrodes may be used for ECG sensing alone or in conjunction with other ECG electrodes (e.g., ECG electrodes on the IPG 210 and/or on the lead 240).

In some examples, the MCU 211 is configured to initiate or modify stimulation provided by the stimulation system (e.g., the stimulation current provided by the driver 213) based on the measured ECG data (e.g., based on a heart rate determined based on the measured ECG data). For example, the MCU 211 may be configured to determine the onset of an epileptic seizure in a patient based on the measured ECG data. Sudden increases in heart rate, or certain patterns of the heart rate, can indicate that a seizure is about to occur. The MCU 211 may calculate and monitor heart rate data based on the ECG data and, in response to determining that a seizure is about to occur based on the heart rate data, initiate stimulation and/or increase stimulation, for example, to the vagus nerve in order to prevent, or to reduce the severity of, the oncoming seizure.

In some examples, the MCU 211 is configured to selectively cause the IPG to operate in a monitoring mode or in a stimulation mode.

During the monitoring mode, the MCU 211 may continuously or periodically monitor the captured ECG signals and/or calculate the heart rate based on the captured ECG signals. In some examples, the MCU 211 does not cause the stimulation system to provide stimulation, for example, by not causing the driver 413 to provide the stimulation current. The MCU 211 may cause the electrical switch 416 to maintain its connection to the second electrical path 218 during the monitoring mode. For example, during the monitoring mode, the electrical switch 416 may not be moved to the first electrical path 217. The MCU 211 may be configured to determine whether the ECG data, or the corresponding heart rate data calculated based on the ECG data, exhibits a set warning behavior. For example, the warning behavior may be a behavior that indicates the onset of a seizure. In some examples, the warning behavior may include an increase in the heart rate over a set time period that is above a threshold value. In some examples, the warning behavior includes any change in the heart rate over the set time period that exhibits a set pattern.

During the stimulation mode, the MCU 211 may cause the stimulation system to provide stimulation, for example, by causing the current driver 413 to provide the stimulation current. The MCU 211 may cause the IPG to operate in the stimulation mode in response to determining that the heart rate value exhibits the warning behavior. In some examples, the MCU 211 may cause the IPG 210 to collect ECG data during the stimulation mode, and the MCU 211 may continuously or periodically monitor the captured ECG signals and/or calculate the heart rate based on the captured ECG signals during the stimulation mode. For example, the MCU 211 may cause the electrical switch 416 to move back and forth between the first and second electrical paths 217 and 218 during the stimulation mode, for example, via a time-division multiplexing operation as described herein. Accordingly, even when the stimulation is providing stimulation, the MCU 211 can continue to collect and monitor ECG data, and the MCU 211 may modify (e.g., increase, decrease, or stop) the stimulation based on said ECG data.

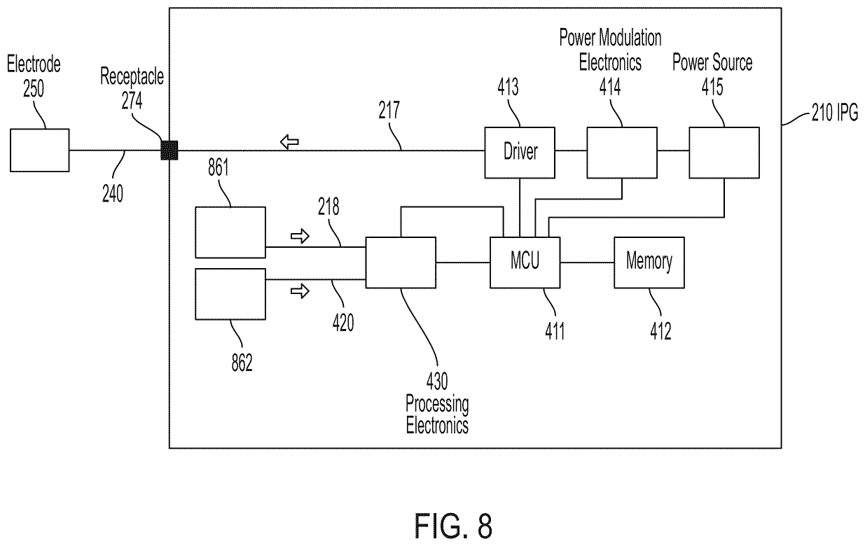

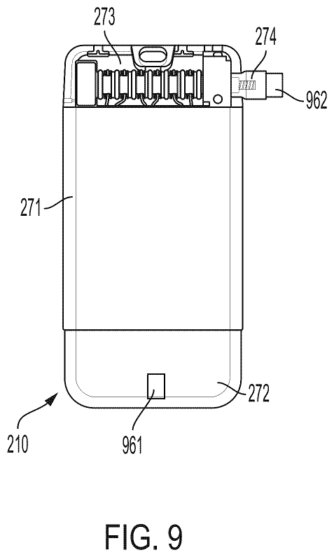

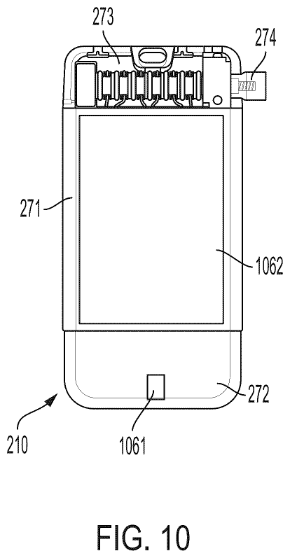

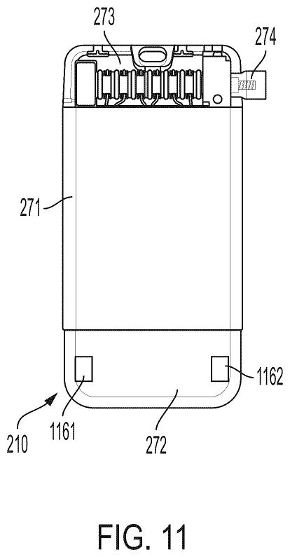

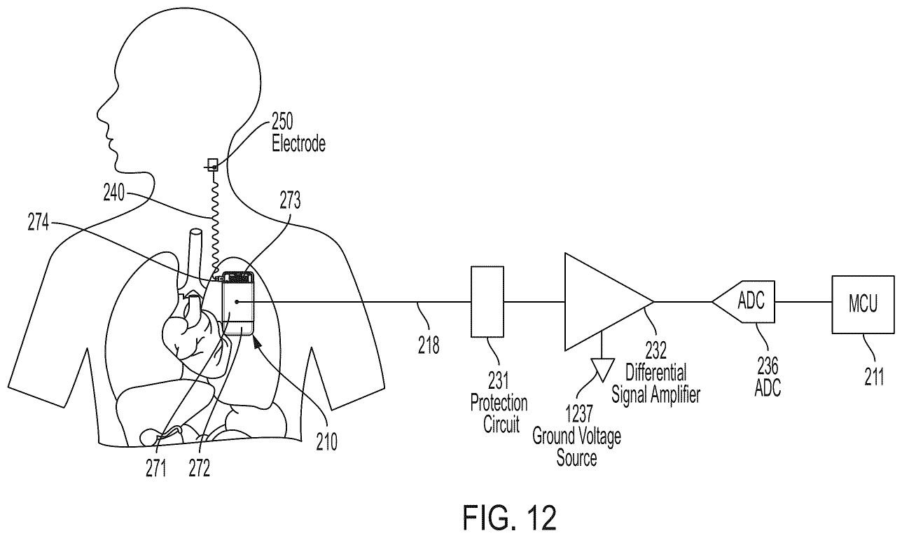

FIG. 7 depicts another stimulation system according to some examples, where the stimulation system is depicted as being implanted in a person and some circuit electronics, contained within stimulator/IPG 210, of the stimulation system are schematically depicted outside of the system for sake of clarity. FIG. 8 depicts a schematic view of some electronic components of the stimulation system of FIG. 7 according to some examples. FIG. 9 depicts the implantable pulse generator (IPG) 210 of the stimulation system of FIG. 7 according to some examples. FIG. 10 depicts the implantable pulse generator 210 of the stimulation system of FIG. 7 according to some other examples. FIG. 11 depicts the implantable pulse generator 210 of the stimulation system of FIG. 7 according to some other examples. FIG. 12 depicts the implantable pulse generator 210 of the stimulation system of FIG. 7 according to some other examples. The stimulation system of FIGS. 7-12, and the methods of operating the stimulation system of FIGS. 7-12, may include features similar to, or the same as, the features of the stimulation system of FIGS. 2-6 and the methods of operating the stimulation system of FIGS. 2-6. Therefore, redundant descriptions may not be provided.

In some examples, the stimulation system of FIG. 7 is not configured for the stimulation electrode 250 to transmit an ECG signal to the MCU 411, and the IPG 210 includes at least two ECG electrodes configured to capture ECG signals and to transmit the captured ECG signals to the processing electronics 430. For example, the IPG 210 may include a first ECG electrode 861 and a second ECG electrode 862, where the first ECG electrode 861 is electrically connected to the processing electronics 430 along the second electrical path 218 and the second ECG electrode 862 is electrically connected to the processing electronics 420 along the fourth electrical path.

The first and second ECG electrodes 861 and 862 may be distinct electrodes that are electrically separated from each other on the outer surface of the IPG 210. Each of the first and second ECG electrodes 861 and 862 may independently include a conductive element on the header 273, a conductive element on the receptacle 274, a conductive element on the insulator portion 272, and/or at least part of the can 271. In some examples, one of the first ECG electrode 861 or the second ECG electrode 862 may include an electrode ring near a proximal portion of the lead 240 that is configured to be adjacent to the receptacle 274 when the lead 240 is coupled to the IPG 210.

The stimulation system of FIGS. 7-8 may be configured to provide stimulation, capture ECG signals, and process the captured ECG signals via the processing electronics 430 in a similar, or same, manner as the stimulation system of FIGS. 2-6. FIGS. 9-12 depict various example stimulation systems that are generally in accordance with FIGS. 7-8 and that have different ECG electrode configurations, which can provide certain advantages as described in more detail below.

Referring to the example of FIG. 9, the IPG 210 may have a first ECG electrode 961 on the insulator portion 272 and a second ECG electrode 962 on the receptacle 274. The first ECG electrode 961 on the insulator portion 272 may be a conductive (e.g., metal) piece on, or exposed to, the outer surface of the insulator portion 272. Depending on how the first ECG electrode 961 is configured (e.g., mounted), the ECG signal could be capacitively coupled into the internal electronics or through a conductive path through the insulator 272. The second ECG electrode 962 may be, for example, a metal ring around the outer surface of the receptacle 274. Because the first and second ECG electrodes 961 and 962 are respectively on the insulator portion 272 and the receptacle 274, the second ECG electrode 962 may be separated from the first ECG electrode by a relatively large distance (e.g., approximately the length of the IPG 210), which can result in a higher quality ECG measurement compared to if the first and second ECG electrodes 961 and 962 were relatively close together. The minimum separation distance for implantable ECG electrodes may be about 35 mm (1.37 inches). For example, the minimum separation distance between the first and second ECG electrodes 961 and 962 may be at least 35 mm, 40 mm, 50 mm, 75 mm, 100 mm, or 200 mm. This distance ensures that there is a sufficient voltage difference between the ECG electrodes to generate an adequate signal-to-noise ratio (SNR) and capture details of the QRS complex, which may be desirable for accurate interpretation of cardiac activities. However, the ECG electrodes could be much closer if only heart rate is to be detected. For example, the minimum separation distance between the first and second ECG electrodes 961 and 962 may be less than 35 mm, 30 mm, 25 mm, 20 mm, 15 mm, 10 mm, 5 mm, or 1 mm.

Referring to the example of FIG. 10, the IPG 210 may include a first ECG electrode 1061 on the insulator portion 272 and a second ECG electrode 1062, which may include at least part of the can 271. The first ECG electrode 1061 may capacitively couple ECG signals into the electronics internal to the IPG (e.g., to the processing electronics). In some examples, the first ECG electrode 1061 may also be an electrical conductor that penetrates through the insulator 272 while maintaining hermeticity. By configuring at least part of the can 271 as an ECG electrode, the ECG electrode can be provided without adding an additional dedicated ECG electrode to the stimulation system.

Referring to the example of FIG. 11, the IPG 210 may include a first ECG electrode 1161 and a second ECG electrode 1162, where both of the first and second ECG electrodes 1161 and 1162 are on the insulator portion 272. The first and second ECG electrodes 1161 and 1162 may be positioned on opposite sides (e.g., on left and right sides, as shown in FIG. 11) of the insulator portion 272, which can increase the distance between the first and second electrodes 1161 and 1162 and improve the quality of the ECG measurement compared to if the first and second ECG electrodes 1161 and 1162 are relatively closer together on the insulator portion 272.

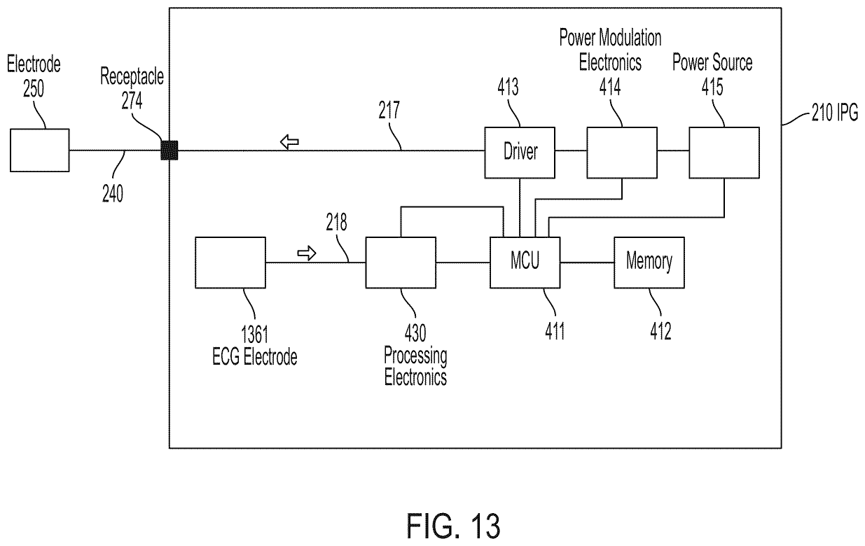

FIG. 12 depicts another stimulation system according to some examples, where the stimulation system is depicted as being implanted in a person and some circuit electronics of the stimulation system are schematically depicted outside of the IPG 201 for sake of clarity, but are placed inside the IPG 201. FIG. 13 depicts a schematic view of some electronic components of the stimulation system of FIG. 12 according to some examples. The stimulation system of FIGS. 12-13 may include features similar to, or the same as, features of the stimulation systems of FIGS. 2-11.

The stimulation system of FIGS. 12-13 is configured to perform monopolar ECG sensing with a single ECG electrode 1361. The ECG electrode 1361 may be on the IPG 210 and may, for example, include at least part of the can 271, a conductive element on the header 273, a conductive element on the receptacle 274, and/or a conductive element on the insulator portion 272. In some examples, the ECG electrode 1361 is an electrode (e.g., a ring electrode) near a proximal portion of the lead 240. In some examples, the ECG electrode 1361 is the stimulation electrode 250, and the lead 240 may be configured to be selectively electrically coupled to the first electrical path 217 or to the second electrical path 218 in a manner similar to, or the same as, the manner described with reference to the stimulation system of FIGS. 2-6.

The differential signal amplifier 232 may be configured to receive a single ECG signal from the ECG electrode 1361 and may generate an output signal based on the ECG signal and a ground voltage from a ground voltage source 1237. For example, the differential signal amplifier 232 may be configured to compare the received ECG signal to the ground voltage to generate the output signal. The ground voltage source 1237 may be a ground voltage on a circuit board (e.g., a printed circuit board) of the differential signal amplifier or of the IPG 210. Electrode 250 may also be connected to the ground voltage of the circuit board to provide a reference voltage at a distant site away from the heart. Monopolar ECG detection may use a single active electrode to measure the potential difference between the active electrode and a reference voltage level (e.g., the board ground in some examples of the medical system of FIGS. 12-13, which is electrically shielded by the can). This method can be simpler in terms of hardware design and implementation, for example, because only one ECG electrode (e.g., electrode 1361) may be needed. On the other hand, bipolar or multipolar ECG detection can allow for better noise removal (e.g., removal of common mode noise). While gross features, such as heart rate, can be detected with monopolar ECG detection, finer waveform features such as P wave and T Wave may be more distinguishable with bipolar or multipolar ECG detection.

Although some methods for providing stimulation and ECG sensing have been discussed herein, the present disclosure is not limited thereto. Stimulation systems for providing stimulation and sensing ECG data, and operations performed by such systems, have been described herein with reference to FIGS. 2-12, and the present disclosure includes all methods for providing stimulation and sensing ECG data that include any combination of such operations in any suitable order.

Each feature of embodiments of the present disclosure may be combined or combined with each other, partially or entirely, and may be technically interlocked and operated in various ways, and each embodiment may be implemented independently of each other or in conjunction with each other.

It will be understood that, although the terms “first”, “second”, “third”, etc., may be used herein to describe various elements and/or features, these elements and/or features should not be limited by these terms. These terms are only used to distinguish one element or feature from another element or feature. Thus, a first element or feature discussed herein could be termed a second element or feature, without departing from the spirit and scope of the present disclosure.

As used herein, the term “and/or” includes any and all combinations of one or more of the associated listed items. The use of “may” when describing embodiments of the present disclosure refers to “one or more embodiments of the present disclosure.”

It will be understood that when an element or feature is referred to as being “on”, “connected to”, “coupled to”, or “adjacent to” another element or feature, it can be directly on, connected to, coupled to, or adjacent to the other element or feature, or one or more intervening element(s) or feature(s) may be present. In contrast, when an element or feature is referred to as being “directly on,” “directly connected to”, “directly coupled to”, or “immediately adjacent to” another element or feature, there are no intervening elements or features present. Similar terms and phrases should be understood in a similar manner to encompass both direct and indirect affiliations between two or more elements or features being discussed. In addition, it will also be understood that when an element or feature is referred to as being “between” two elements or features, it can be directly between the two elements or features (e.g., it can be the only element or feature between the two elements or features), or one or more intervening elements or features may also be present.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The stimulation systems and/or any other relevant devices or components according to embodiments of the present disclosure described herein may be implemented utilizing any suitable hardware, firmware (e.g. an application-specific integrated circuit), software, or a combination of software, firmware, and hardware. For example, the various components of the stimulation systems may be formed on one integrated circuit (IC) chip or on separate IC chips. Further, the various components of the stimulation systems may be implemented on a flexible printed circuit film, a tape carrier package (TCP), a printed circuit board (PCB), or formed on one substrate. Further, the various components of the stimulation systems may be a process or thread, running on one or more processors, in one or more computing devices, executing computer program instructions and interacting with other system components for performing the various functionalities described herein. The computer program instructions may be stored in a memory which may be implemented in a computing device using a standard memory device, such as, for example, a random access memory (RAM). The computer program instructions may also be stored in other non-transitory computer readable media. Also, a person of skill in the art should recognize that the functionality of various computing devices may be combined or integrated into a single computing device, or the functionality of a particular computing device may be distributed across one or more other computing devices without departing from the scope of the example embodiments of the present disclosure.

Although specific embodiments are described herein, the scope of the technology is not limited to those specific embodiments. Moreover, while different examples and embodiments may be described separately, such embodiments and examples may be combined with one another in implementing the technology described herein. One skilled in the art will recognize other embodiments or improvements that are within the scope and spirit of the present technology. Therefore, the specific structure, operations, and methods are disclosed only as illustrative embodiments. The scope of the technology is defined by the following claims and any equivalents therein.

What is claimed is:

1. A medical system, comprising:
an implantable pulse generator (IPG);
a lead electrically coupled to the IPG; and
an electrode on the lead, the electrode being configured to provide a stimulation signal to, and receive a first electrocardiogram (ECG) signal from, tissue around the electrode,
wherein the IPG comprises a current source configured to provide an alternating current through the lead to the electrode, and
wherein the lead is configured to selectively receive the current from the current source or to transmit the first ECG signal.

2. The medical system of claim 1, wherein the IPG comprises a microcontroller configured to control of configuration of the lead and to determine at least one of a heart rate value, a respiration rate value, or a respiration phase, based on the first ECG signal.

3. The medical system of claim 2, wherein the IPG further comprises a memory storing instructions that, when executed by the microcontroller, cause the microcontroller to change, at a set frequency, the configuration of the lead between a first configuration, whereby the lead is electrically coupled to the current source and receives the current, and a second configuration, whereby the lead is electrically coupled to the microcontroller and transmits the first ECG signal.

4. The medical system of claim 2, further comprising an electrical switch that is electrically coupled to the lead and positionable at least in a first position, whereby the lead is electrically coupled to the current source along a first electrical path, and a second position, whereby the lead is electrically coupled to the microcontroller along a second electrical path different from the first electrical path.

5. The medical system of claim 4, wherein the IPG further comprises, along the second electrical path, at least one of:
a protection circuit configured to remove or attenuate signals having a voltage above a threshold voltage, or
a differential signal amplifier configured to receive the first ECG signal, to receive a second ECG signal, and to generate a differential ECG signal based on the first and second ECG signals, and
wherein the microcontroller is configured to determine the at least one of the heart rate value, the respiration rate value, or the respiration phase, based on the differential ECG signal.

6. The medical system of claim 2, wherein the microcontroller is configured to cause the lead to alternatingly receive the current from the current source and to transmit the first ECG signal at a frequency.

7. The medical system of claim 2, wherein the lead is configured to transmit the first ECG signal to the microcontroller.

8. The medical system of claim 2, further comprising:
a protection circuit configured to receive the first ECG signal and to remove or attenuate signals having a voltage above a threshold voltage; and
a differential signal amplifier configured to receive the first ECG signal, to receive a second ECG signal, and to generate and output a differential ECG signal based on the first and second ECG signals.

9. The medical system of claim 8, wherein the lead is configured to transmit the first ECG signal to the protection circuit, and the differential signal amplifier is electrically coupled between the protection circuit and the microcontroller.

10. The medical system of claim 8, wherein the differential signal amplifier is further configured to amplify a magnitude of the differential ECG signal.

11. The medical system of claim 8, wherein the IPG further comprises a low-pass filter and a high-pass filter that are respectively configured to filter out signals that have a frequency below a lower threshold frequency and that have a frequency above an upper threshold frequency.

12. The medical system of claim 2, wherein the IPG further comprises a conductive can configured to house electronic components of the IPG and to receive a second ECG signal from tissue around the conductive can, and
wherein the microcontroller is configured to determine the at least one of the heart rate value, the respiration rate value, or the respiration phase, based on the first and second ECG signals.

13. The medical system of claim 2, wherein the IPG further comprises a header configured to receive the lead and comprising a conductive element configured to receive a second ECG signal from tissue around the header, and
wherein the microcontroller is configured to determine the at least one of the heart rate value, the respiration rate value, or the respiration phase, based on the first and second ECG signals.

14. The medical system of claim 2, wherein the IPG further comprises a memory storing instructions that, when executed by the microcontroller, cause the IPG:
to operate in a monitoring mode, in which the microcontroller determine the heart rate value based on the first ECG signal and determines whether the heart rate value exhibits a warning behavior; and
to operate, in response to determining that the heart rate value exhibits the warning behavior, in a stimulation mode in which the current source provides the current through the lead to the electrode.

15. The medical system of claim 14, wherein the warning behavior comprises an increase in the heart rate value over a set time period that is above a threshold value.

16. The medical system of claim 2, wherein the electrode is a cuff electrode configured to stimulate a vagus nerve.

17. The medical system of claim 2, wherein the microcontroller is configured to calculate at least the heart rate value based on the first ECG signal.

18. The medical system of claim 17, wherein the microcontroller is further configured to calculate at least the respiration rate value based on the first ECG signal.

19. The medical system of claim 2, wherein the microcontroller is configured to calculate at least the respiration rate value based on the first ECG signal.

20. An implantable pulse generator (IPG), comprising:
a driver configured to generate an alternating stimulation current;
a conductive can configured to house electronic components of the IPG and to receive a first electrocardiogram (ECG) signal from tissue around the conductive can;
a protection circuit configured to receive the first ECG signal and to remove or attenuate signals having voltages above a threshold voltage;
a differential signal amplifier configured to receive the first ECG signal, receive a second ECG signal, and generate an output signal based on a difference between the first and second ECG signals; and
a microcontroller configured to receive the output signal.

21. The IPG of claim 20, wherein the microcontroller is configured to determine, based on the output signal, at least one of a heart rate value, a respiration rate value, or a respiration phase.

22. The IPG of claim 21, wherein the microcontroller is configured to determine at least the heart rate value.

23. The IPG of claim 20, wherein the differential signal amplifier is further configured to amplify the output signal.

24. The IPG of claim 20, further comprising at least one of:
a low-pass filter configured to filter out signals having a frequency below an upper threshold frequency; or
a high-pass filter configured to filter out signals having a frequency above a lower threshold frequency.

25. The IPG of claim 20, further comprising a header configured to receive, and electrically connect to, a lead,
wherein the differential signal amplifier is configured to receive the second ECG signal from the header.

26. The IPG of claim 20, wherein the differential signal amplifier is configured to receive the second ECG signal from a conductive element on the IPG that is electrically insulated from the conductive can.

27. A medical system, comprising:
the IPG of claim 20;
a lead electrically coupled to the IPG; and
an electrode on the lead that is configured to stimulate tissue around the electrode.

28. A medical system, comprising:
an implantable pulse generator (IPG);
a lead electrically coupled to the IPG; and
a stimulation electrode on the lead,
wherein the IPG comprises:
a current source configured to provide an alternating current through the lead to the stimulation electrode;
a conductive can configured to house electronic components of the IPG and to receive a first electrocardiogram (ECG) signal from tissue around the conductive can; and
an ECG electrode electrically insulated from the conductive can and configured to receive a second ECG signal from tissue around the ECG electrode.

29. The medical system of claim 28, wherein the ECG electrode is a conductive element on a header of the IPG that is configured to receive, and electrically connect to, the lead.

30. The medical system of claim 28, wherein the IPG further comprises a microcontroller configured to determine at least one of a heart rate value, a respiration rate value, or a respiration phase, based on the first and second ECG signals.

31. The medical system of claim 30, wherein the IPG further comprises at least one additional ECG electrode respectively configured to receive at least one additional ECG signal, and
wherein the microcontroller is configured to determine the at least one of the heart rate value, the respiration rate value, or the respiration phase, based on the first ECG signal, the second ECG signal, and the at least one additional ECG signal.

32. The medical system of claim 30, wherein the microcontroller is configured to determine at least the heart rate value based on the first and second ECG signals.

33. The medical system of claim 30, wherein the microcontroller is configured to determine at least the respiration rate value based on the first and second ECG signals.
